(19) United States
(12) Patent Application Publication
(10) Pub. No.: US 2022/0087740 A1
(43) Pub. Date: Mar. 24, 2022

(54) MULTI-ELECTRODE ABLATION DEVICE

(71) Applicant: SYMAP MEDICAL (SUZHOU), LTD, Suzhou, Jiangsu (CN)

(72) Inventor: Jie WANG, Suzhou, Jiangsu (CN)

(21) Appl. No.: 17/425,326

(22) PCT Filed: Jan. 30, 2020
(86) PCT No.: PCT/CN2020/074075

§ 371 Date: Jul. 23, 2021

(30) Foreign Application Priority Data
Jan. 30, 2019 (CN) 201910089932.0
Jan. 31, 2019 (CN) 201910096490.2

(51) Int. Cl. A61B 18/14; A61B 18/12

(52) U.S. Cl. CPC A61B 18/1492; A61B 2018/00261; A61B 18/1233

(57) ABSTRACT

A device for radio frequency ablation, configured to deliver a direct current, an alternating current, and a radio frequency energy to a lesion for treating a pulmonary disease. The device for radio frequency ablation can determine the effectiveness of an ablation according to one or more of a fall in impedance, a rate of change in impedance, a change in the rate of change in impedance, or a change from falling in impedance to rising in impedance. The device for radio frequency ablation uses a segmentation control method and dynamic smoothing for adjusting a radio frequency output power to control an ablation temperature, and the tissue to be ablated is prevented from being quickly heated in short time, to ensure a smooth change in the radio frequency output power in the ablation process. The device for radio frequency ablation further comprises a specific protection mechanism for preventing repeated ablation. The temperature of an ablation site is detected before each ablation, and ablation will not be performed if the temperature of the ablation site is higher than 40° C. to 60° C. Also disclosed is a multi-electrode ablation device comprising the device for radio frequency ablation.

### FIELD OF THE INVENTION

The present invention belongs to the field of a minimally invasive medical apparatus, and particularly relates to a device for radio frequency ablation and a multi-electrode ablation device for delivering energy in the trachea and bronchus.

### BACKGROUND OF THE INVENTION

Chronic obstructive pulmonary disease is a progressive disease which may cause obstruction of the lung airways, restricting airflow from going in and out of the lungs, such as asthma, emphysema, and chronic obstructive pulmonary diseases. Therefore, patients with the chronic obstructive pulmonary disease have difficulty in breathing and have symptoms such as coughing, wheezing, shortness of breath, chest tightness, and mucus (asthma attacks), requiring clinical treatment and consuming a lot of medical resources, and may result in hospitalization and life-threatening danger. The causes of the chronic obstructive pulmonary disease are as follows: airway smooth muscle contraction, secretion of too much mucus by the airway glands, thickening of the airway wall smooth muscles due to inflammation, and changes of anatomical structures of the tissues around the airway.

Pathological hyperplasia as well as excessive and inappropriate contraction of the airway smooth muscles in the lung airway wall of the patients is one of pathological mechanisms of the chronic obstructive pulmonary disease. Therefore, reducing or eliminating pathologically hyperplastic airway smooth muscles is an option for treating the chronic obstructive pulmonary disease.

At present, the main method for clinically treating the chronic obstructive pulmonary diseases such as asthma, emphysema and chronic obstructive pulmonary disease is through medicine treatments such as adrenaline medication, theophylline medication and hormones, or symptomatic treatment such as sputum excretion, anti-inflammation and the like, which requires long-term medication, and cannot cure this type of diseases. Moreover, some patients are still unable to effectively control their condition after using inhaled corticosteroids (ICS) and long-acting β receptor agonists (LABA).

An existing minimally invasive ablation technique can reduce the pathologically hyperplastic airway smooth muscles. During the implementation of this treatment, a catheter is positioned in the airway, and electrode arrays on the tail end of the catheter expand to contact with the airway wall. By moving the catheter, energy is gradually delivered to multiple parts of the trachea to remove the pathologically hyperplastic airway smooth muscles.

The safety and effectiveness of ablation apparatus used in bronchial radio frequency ablation based on the prior art have limitations, for example, the contact status between the ablation electrodes and the wall cannot be monitored and displayed; and at the moment when the ablation starts, a relatively large radio frequency energy is applied, resulting in a large temperature overshoot after the set temperature is reached, such suddenly applied and (or) suddenly changed radio frequency energy has an irritating effect on the respiratory tract of the patient, and the large temperature overshoot threatens the safety of the patient. Additionally, in the treatment process of the bronchial radio frequency ablation, the temperature of ablation electrodes is affected by frequent and complicated disturbance due to the change of the airflow caused by the breathing movement of the patient, the sliding of the electrodes caused by the chest movement of the patient, and the change of the level of contact caused by unstable grip of the surgeon, thus the general proportional integral control algorithm can easily generate oscillation and overshooting, and is difficult to adapt to these complicated external disturbances, as such the ablation treatment effect is interfered.

The ideal bronchial radio frequency ablation should avoid repeated ablation at the same site. However, due to the carelessness or incorrect operation of the surgeon, or the lack of a reminder function provided in the apparatus used during the actual clinic operation, after one ablation is completed, and the ablation is started again without catheter (electrode) transferring or with insufficient catheter transfer, repeated ablation at the same site will occur, resulting in permanent and irreversible damage to the airway tissues, or even airway fistula. The present invention adopts control mechanisms such as defining the logic relationship among the impedance, power and temperature, and detecting the temperature of an ablation site before each ablation, wherein ablation will not be performed if the temperature of ablation site is higher than 40° C. to 60° C., preferably 45° C., so that the device for radio frequency ablation of the present invention has a protection mechanism of preventing repeated ablation.

### SUMMARY OF THE INVENTION

An objective of the present invention is to target the limitations in the prior art, providing a safer and more effective device for delivering energy in the trachea and bronchus.

In order to achieve the above objective, the present invention adopts the following technical solutions.

A device for radio frequency ablation is able to generate and control direct current, alternating current and radio frequency energy, collect, process and display a temperature, impedance or tension signal, and determine effectiveness of an ablation according to change of impedance or tension signal, said change of impedance is one or more parameters selected from the group consisting of fall in impedance, rate of change in impedance, a change in rate of change in impedance, and a change from falling in impedance to rising in impedance.

Further, the ablation is determined to be effective when a fall in impedance exceeds 10Ω to 100Ω, or the rate of change in impedance is higher than −1 Ω/s to −50 Ω/s, or the impedance changes from falling in impedance to rising in impedance.

Further, the ablation is determined to be effective when the fall in the value of impedance exceeds 20Ω to 50Ω, or the rate of change in impedance is higher than −5 Ω/s to −50 Ω/s, or the impedance changes from falling in impedance to rising in impedance.

Further, said device for radio frequency ablation uses a segmentation control method via a closed-loop control system to adjust a radio frequency output power so as to control an ablation temperature, said segmentation control comprises: (1) a fast heating phase: lasting for 0.5 s to 2 s from the beginning of ablation to reach a fast heating phase end point temperature that is 50% to 80%, preferably 65% of the ablation temperature; (2) a slow heating phase: lasting for 0.5 s to 2 s after the fast heating phase to reach a slow heating phase end point temperature that is 70% to 99%, preferably 90% of the ablation temperature, or is 0.1° C. to 10° C., preferably 2° C. lower than the ablation temperature; and (3) a stable maintenance phase: the temperature is stably maintained after the slow heating phase until the ablation is stopped.

Each of the above phases can be respectively optimized according to inherent characteristics of an apparatus in accordance with practical treatment requirements. In the whole ablation treatment process, the radio frequency output power starts to smoothly change from 0. At the fast heating phase, the radio frequency output power rises fast, at the slow heating phase, the radio frequency output power gradually and slowly rises and is then changed into slow falling, and at the stable maintenance phase, the radio frequency output power continuously and slowly falls to gradually become stable.

The level of contact between each treatable site of the bronchus and different electrodes are determined by using a segmentation proportional integral control algorithm, and a bronchial device for radio frequency ablation can control the radio frequency output power so that the temperature of ablation electrodes can reach a set temperature within 3 s, and additionally, the temperature overshoot after the set temperature is reached is less than 3° C., and generally ranges from 0.5° C. to 1.5° C. The temperature is stably maintained at the set temperature, with a fluctuation smaller than 1° C., and is generally smaller than 0.5° C. In the whole ablation treatment process, the radio frequency output power smoothly changes without suddenly applied and (or) suddenly changed radio frequency energy.

Further, the device for radio frequency ablation of the present invention performs dynamic smoothing on the temperature in the process of controlling the ablation temperature, comprising averaging, weighted averaging or median averaging sampled temperature values; said device for radio frequency ablation is guided to adjust the radio frequency power output based on said dynamic smoothed temperature values , to ensure smooth change of the radio frequency output power during the ablation process.

An upper threshold value of said dynamic smoothing is 0.1° C./s to 20° C./s, preferably 5° C./s, and a lower threshold value is −0.1° C./s to −20° C./, preferably −5° C./s, when a temperature change rate is smaller than the lower threshold value, the smoothing time window is prolonged; when the temperature change rate is greater than the upper threshold value, the smoothing time window is shortened; and when the temperature change rate is between the lower and the upper threshold value, the smoothing time window remains unchanged

The smoothing time window has a dynamic range of preferably 0 s to 10 s, more preferably 0 s to 2.5 s.

The above temperature dynamic smoothing can adapt to various complicated disturbances, the radio frequency output power smoothly changes in the whole ablation treatment process, sudden changes in the radio frequency energy does not occur, the temperature is stable, the fluctuations are very small, and oscillation and overshoot cannot occur even if very frequent and very violent disturbance occurs. Additionally, temperature overshoot possibly caused by disturbance can be well inhibited, and the temperature overshoot cannot exceed 3° C. even with violent and complicated disturbance. Therefore, safety and effectiveness of the process for removing pathological hyperplasia by ablation energy are ensured.

Further, the device for radio frequency ablation of the present invention further comprises a protection mechanism for preventing repeating ablation. The temperature of an ablation site is detected before each ablation. Ablation will not be performed if the temperature of the ablation site is higher than 40° C. to 60° C., preferably 45° C.

Further, said device for radio frequency ablation uses one or both of the following methods: Method 1: detecting the impedance by a continuous weak alternating current signal, and calculating the impedance through a voltage and a current during radio frequency output; Method 2: directly detecting the impedance without radio frequency output.

Further, said device for radio frequency ablation comprises a radio frequency energy delivery/feedback control mechanism: radio frequency energy is delivered to a tissue for 2 to 4 s so that said tissue reaches and maintains a set temperature for 6 to 8 s, and when the temperature of said tissue is higher than an over-temperature threshold value, an over-temperature alarm will be triggered and said device will stop delivery of radio frequency energy. Preferably, said set temperature ranges from 60° C. to 70° C., and said over-temperature threshold value is 1° C. to 10° C. higher than the set temperature.

More preferably, said set temperature is 65° C., and the over-temperature threshold value is 3° C. higher than the set temperature.

Further, said device for radio frequency ablation adopts a design of multiple central controllers, dual circuit design for temperature, voltage and current.

Further, said device for radio frequency ablation comprises a data transmission interface for external connection to a computer to obtain information of various parameters (such as temperature, impedance, power, time and whether the ablation succeeds or not) in real time.

Further, said device for radio frequency ablation comprises a touch display screen for displaying a status of the electrodes and a contact impedance value between the electrodes and a tissue, and energy can be delivered from one or more electrodes by clicking the touch display screen.

Another objective of the present invention is to provide a multi-electrode ablation device, comprising the device for radio frequency ablation of the present invention, electrode assemblies, a guiding catheter, a handle and a connector.

Said guiding catheter comprises at least one lumen;
said electrode assemblies are disposed at a front end of the guiding catheter, and is connected to the handle through circuitries inside said guiding catheter, said electrode assemblies comprise more than one electrode groups and more than one detection devices, said electrode groups are able to deliver electric energy, radio frequency energy, laser energy, high-density focused ultrasound or low temperature for ablation, and said detection devices are configured to detect temperature, impedance or tension;
said handle is connected to the connector and said electrode assemblies, and comprises one or more operation components, said operation components are configured to control constriction, expansion and energy release of the electrode groups and are able to control the electrode assemblies to extend out of or retreat back into the guiding catheter; and
said connector is configured to provide an energy to the electrodes.

Further, said device for radio frequency ablation displays an impedance or tension of the electrodes and indicate whether the electrode assemblies are in good contact with the tracheal wall: an impedance value smaller than or equal to a threshold value of impedance after the electrodes are in contact with the tissue indicates good contact between said electrode assembly and the tracheal wall.

Preferably, said threshold value of impedance ranges from 500Ω to 1000Ω, more preferably 900Ω.

Further, a method for determining whether the electrodes are in good contact with the tracheal wall by said device for radio frequency ablation is as follows: the device for radio frequency ablation can determine the impedance of each electrode, and if the impedances are consistent, the contact between the electrodes and the tracheal wall is good; if the contact between a certain electrode and the tracheal wall is not good, the impedance is different from that of others in good contact.

Further, said device for radio frequency ablation simultaneously uses two impedance detection methods: Method 1: detecting the impedance by a continuous weak alternating current signal, and calculating the impedance through a voltage and a current during radio frequency output; and Method 2: directly detecting the impedance without radio frequency output.

Further, the detection device comprises a temperature detection device, an impedance detection device and a tension detection device.

Further, the electrode group comprises one or more electrodes, each electrode is electrically connected to the handle independently, the electrode groups expands in a basket shape, spiral shape or balloon shape under the control of the operation components, and under the presence of more than one electrode groups, the electrode groups are sequentially arranged in series with electrode groups closer to the handle having a larger outer diameter after expansion, and said outer diameter is from 1 to 20 mm.

Further, the electrode assemblies further comprise a steel wire, each of said electrodes comprises two ends, each of said two ends of is fixed to the steel wire, said steel wire passes through the guiding catheter to be connected to the handle, and the handle controls the contraction and expansion of the electrode groups by pulling and releasing the steel wire.

Further, in the presence of more than one electrode group, a damage-prevention structure is disposed at a tip of an electrode group that is most distal from the handle among said electrode groups, and the electrode groups are connected to each other through support components.

Further, a pressure sensor is disposed on the steel wire.

Further, the electrode assembly further comprises a balloon, said balloon is disposed between the electrodes, said balloon is connected to said handle via a balloon air passage which passes through said guiding catheter, said balloon is adapted for connection to an gas inlet apparatus through the handle, and the electrode group expands after the balloon is inflated; under the presence of more than one electrode group, more than one balloons are sequentially arranged in series, and are respectively connected to the handle through independent balloon air passages.

Further, is the hardness of said guiding catheter increases with proximity to said handle, said hardness ranges from 90 A to 80 D on the Shore hardness scale.

Further, the operation component of the handle comprises a control circuit board and control buttons, said control circuit hoard is connected to the electrode assemblies and the control buttons, and said control buttons control different components in different electrode assemblies respectively.

Further, said operation component of said handle controls said electrode groups so as to control said one or more electrodes for energy delivery.

In order to achieve the objective of the present invention, the present invention provides a multi-electrode ablation device configured to achieve a function of delivering energy in the trachea and bronchus, mainly comprising a first electrode assembly, a second electrode assembly, a guiding catheter body, a handle and a connector. The first electrode assembly and the second electrode assembly are continuously disposed in an axial direction of the guiding catheter body, a damage-prevention structure is disposed at a tip of the electrode assembly and is configured to fix the first electrode assembly at the same time. The first electrode assembly and the second electrode assembly are connected to each other through a support component, a proximal end of the first electrode assembly and a distal end of the second electrode assembly are fixed to the support component, a distal end of a steel wire is connected to the damage-prevention structure at the tip, and the proximal end is fixed to the support component and enters the handle through the guiding catheter body. The proximal end of the second electrode assembly is fixed to the catheter body, When the handle controls the steel wire to contract towards the proximal end, the first electrode assembly is driven to expand first, at the same time, the second electrode assembly synchronously expands, and according to the characteristics of the trachea tract, the electrode assembly is designed to be smaller at distal end and larger at proximal end, with a diameter difference of about 1 to 5 mm.

The first electrode assembly and the second electrode assembly are provided with a plurality of electrodes: a first electrode, a second electrode, a third electrode, a fourth electrode, a fifth electrode, a sixth electrode, a seventh electrode and an eighth electrode, the electrodes are made of stainless steel materials, and have certain elasticity, each electrode is electrically connected to the handle independently, and the handle is connected to the bronchial device for radio frequency ablation through the connector. When in use, each electrode forms a loop with a control circuit board through a trachea tissue, and each electrode can independently detect a contact impedance value between the electrode and the tissue. When the electrode is in good contact (the detected impedance value is 500Ω to 1000Ω or below), the bronchial device for radio frequency ablation will deliver radio frequency energy to ablate the lesion tissue, each of the first electrode assembly and the second electrode assembly is provided with a temperature sensor, which can independently detect a temperature of the tissue around the corresponding electrode assembly.

Or, a first balloon and a second balloon are disposed under the first electrode assembly and the second electrode assembly, a proximal end of the first balloon is provided with a first balloon air passage, and a proximal end of the second balloon is provided with a second balloon air passage. The first balloon and the second balloon are isolated from each other, and the first air passage and the second air passage independently provide gas for the first balloon and the second balloon. When the gas enters the balloons through the balloon air passages, the first electrode, the second electrode, the third electrode, the fourth electrode, the fifth electrode, the sixth electrode, the seventh electrode and the eighth electrode expand under pressure, the electrode assemblies expand, the gas inflow is controlled by an external gas inlet apparatus, the expansion size of the electrode assemblies can be set through the gas inflow, and the first electrode assembly and the second electrode assembly are independently controlled to adapt to the requirements of different sizes of the trachea lesion sites. The first electrode, the second electrode, the third electrode, the fourth electrode, the fifth electrode, the sixth electrode, the seventh electrode and the eighth electrode are provided with independent conductive wires. When in use, each electrode forms a loop with the control circuit board through the trachea tissue, and each electrode can independently detect the contact impedance value between the electrode and the tissue. A temperature sensor is disposed on each of the electrode assemblies, and can independently detect the temperature of the tissue around the corresponding electrode assembly.

Annular electrodes can instead be used. A first annular electrode and a second annular electrode are spirally disposed on the first balloon and the second balloon. When the balloons are inflated, outer diameters of the first annular electrode and the second annular electrode are increased. Independent conductive wires are disposed on the first annular electrode and the second annular electrode. When in use, each electrode forms a loop with the control circuit board through the trachea tissue, and each electrode can independently detect the contact impedance value between the electrode and the tissue. A temperature sensor is disposed on each of the annular electrodes, and can independently detect the temperature of the tissue around the corresponding electrode assembly.

As a preferable solution of the present invention, an indicating lamp is disposed on the handle. Theoretically, an impedance value of 500Ω to 1000Ω or below after the electrode is in contact with the tissue indicates that the radio frequency ablation can be performed. When the bronchial device for radio frequency ablation detects that the electrode contact impedance value is 500Ω to 1000Ω or below, the indicating lamp becomes green, indicating that the ablation can be performed. When the bronchial device for radio frequency ablation detects that the electrode contact impedance value is 500Ω to1000Ω or above, the indicating lamp is red, indicating that the ablation cannot be performed.

As a preferable solution of the present invention, a pressure sensor is disposed in a local area of the steel wire, the two ends of the pressure sensor are respectively connected to two ends of the steel wire, when the electrode assembly is dragged, the steel wire is stressed. At this moment, the pressure sensor will receive the same tension, and through the treatment by the bronchial device for radio frequency ablation, the tension will be displayed to determine the level of contact. When the electrode is in contact with the tissue, the level of contact between an electrode arm and the tissue can be determined through determining the traction tension.

As a preferable solution of the present invention, the device for radio frequency ablation is provided with a touch display screen for displaying a status of the electrodes and a contact impedance value between the electrodes and a tissue, and energy can be delivered from one or more electrodes by clicking the touch display screen.

As a preferable solution of the present invention, the guiding catheter body can be served as a guiding tube, the guiding tube is provided with a tube lumen accommodating the electrode assembly, the electrode assembly can freely extend and retract in the guiding tube, and liquids, such as anti-inflammatory medicine and anesthetics can enter the ablated lesion tissue through the tube lumen of the guiding tube so as to relive the pain and complications of a patient.

Another objective of the present invention is to provide a method for determining effectiveness of radio frequency ablation, comprising: delivering electrical stimulation to an ablation site, detecting, collecting and processing an impedance value of the ablation site, and determining the effectiveness of the ablation according to change of impedance, wherein said change of impedance is one or more parameters selected from the group consisting of a fall in impedance, rate of change in impedance, a change in the rate of change in impedance, and a change from falling in impedance to rising in impedance.

Preferably, the ablation is determined to be effective when said fall in impedance exceeds 10Ω to 100Ω, more preferably 20Ω to 50Ω, or the rate of change in impedance is higher than −1 Ω/s to −50 Ω/s, more preferably −5 Ω/s to −50 Ω/s, or the impedance changes from falling in impedance to rising in impedance.

Another objective of the present invention is to provide a method for controlling a radio frequency ablation temperature: a segmentation control method is used via a closed-loop control system to adjust a radio frequency output power so as to control the ablation temperature, said segmentation control comprises: (1) a fast heating phase: lasting for 0.5 s to 2 s from the beginning of ablation to reach a fast heating phase end point temperature that is 50% to 80% of the ablation temperature; (2) a slow heating phase: lasting for 0.5 s to 2 s after the fast heating phase to reach a slow heating phase end point temperature that is 70% to 99% of the ablation temperature, or is 0.1° C. to 10° C. lower than the ablation temperature; and (3) a stable maintenance phase: the temperature is stably maintained after the slow heating phase until the ablation is stopped.

Preferably, said segmentation control comprises: (1) a fast heating phase: lasting for 1 s from the beginning of ablation to reach a fast heating phase end point temperature that is 65% of the ablation temperature; (2) a slow heating phase: lasting for 1 s after the fast heating phase to reach a slow heating phase end point temperature that is 90% of the ablation temperature, or is 2° C. lower than the ablation temperature; and (3) a stable maintenance phase: the temperature is stably maintained after the slow heating phase until the ablation is stopped.

Another objective of the present invention is to provide an anti-interference method for a radio frequency ablation temperature, comprising: performing dynamic smoothing on the temperature in the process of controlling the ablation temperature, comprising averaging, weighted averaging or median averaging sampled temperature values; said device for radio frequency ablation is guided to adjust the radio frequency power output based on said dynamic smoothed temperature values to ensure a smooth change of the radio frequency output power during the ablation process.

Preferably, an upper threshold value of the dynamic smoothing is 0.1° C./s to 20° C./s, more preferably 5° C./s, and a lower threshold value is −0.1° C./s to −20° C//, more preferably −5° C./s; when a temperature change rate is smaller than the lower threshold value, the smoothing time window is prolonged; when the temperature change rate is greater than the upper threshold value, the smoothing time window is shortened; and when the temperature change rate is between the lower and the upper threshold value, the smoothing time window remains unchanged.

As a preferable solution of the above method, when the temperature change rate is greater than 1° C./s to 50° C./s, the smoothing time window ranges from 0 s to 10 s. More preferably, when the temperature change rate is greater than 20° C/s, the smoothing time window is 2.5 s.

Another objective of the present invention is to provide a method for preventing repeated ablation, comprising: detecting a temperature of an ablation site before each ablation, and not performing ablation if the temperature of the ablation site is higher than 40° C. to 60° C., preferably 45° C.

### Advantages of the Present Invention

(1) Through defining a logic relationship among the impedance, power and temperature, the present invention precisely controls the generated and controlled direct current, alternating current and radio frequency energy; the temperature, impedance or tension signal is collected, processed and displayed, and the effectiveness of an ablation is determined according to the change of the impedance or tension signal, wherein the change of the impedance is one or more parameters selected from the group consisting of fall in impedance, rate of change in impedance, a change in the rate of change in impedance, and a change from falling in impedance to rising in impedance. The radio frequency output power is adjusted by using the closed loop control system through a segmentation control method to control the ablation temperature, and the temperature dynamic smoothing is utilized to counter various kinds of disturbances. Therefore, the safety and the effectiveness of the present system are further ensured, i.e., situations of incorrect ablation or ablation incapability cannot occur, and the situation of repeated ablation or excessive ablation also cannot occur.

(2) According to the present invention, the level of contact between each treatable site of the bronchus and the different electrodes are determined by using a segmentation proportional integral control algorithm, and the bronchial device for radio frequency ablation can control the radio frequency output power so that the temperature of ablation electrodes can reach the ablation temperature within 3 s; additionally, the temperature overshoot after the ablation temperature is reached is less than 3° C., generally ranging from 0.5° C. to 1.5° C. The temperature is stably maintained at the ablation temperature, and the fluctuation is smaller than 1° C., and is generally smaller than 0.5° C. During the whole ablation treatment process, the radio frequency output power smoothly changes without suddenly applied and (or) suddenly changed radio frequency energy.

(3) According to the present invention, the above temperature dynamic smoothing can adapt to various complicated disturbances, and the radio frequency output power smoothly changes throughout the whole ablation treatment process without a sudden change in the radio frequency energy, the temperature keeps stable, the fluctuation is very small, and oscillation and overshoot cannot occur even if very frequent and very violent disturbances occur. Additionally, temperature overshoot possibly caused by disturbance can be well inhibited, and the temperature overshoot cannot exceed 3° C. even with violent and complicated disturbances.

(4) The device for radio frequency ablation of the present invention further comprises a protection mechanism for preventing repeated ablation. The temperature of an ablation site is detected before each ablation. Ablation will not be performed if the temperature of the ablation site is higher than 40° C. to 60° C. The repeated ablation of the same site due to carelessness or incorrect operation of a surgeon can be simply and effectively avoided.

The present invention provides a device with a function of delivering energy in the trachea and bronchus. The device can be used for delivering a direct current, an alternating current, and a radio frequency energy to a lesion, so as to remove pathologically hyperplastic bronchial smooth muscles, increase the diameter of the trachea during resting, reduce the pathological retraction and respiratory resistance of the tracheal wall, and increase the adjusting compliance of the trachea. The device can be used for the non-medicinal treatment of obstructive pulmonary diseases, and for example, used for the treatment of patients with persistent asthma, pulmonary emphysema, chronic obstructive pulmonary diseases, etc. that are still incapable of being effectively controlled after the administration of medicine (such as corticosteroids and long-acting β receptor agonists)

### BRIEF DESCRIPTION OF THE DRAWINGS

FIG. 1 is an overall schematic diagram of the multi-electrode ablation device of Example 1.

FIG. 2 shows a schematic diagram of the basket-shaped electrode assembly of Example 1 in the unexpanded state.

FIG. 3 shows a first schematic diagram of the basket-shaped electrode assembly of Example 1 in the expanded state.

FIG. 4 shows a second schematic diagram of the basket-shaped electrode assembly of Example 1 in the expanded state.

FIG. 5 shows a local sectional view of a support component in Example 1.

FIG. 6 shows a first schematic diagram of the balloon electrode assembly of Example 2.

FIG. 7 shows a second schematic diagram of the balloon electrode assembly of Example 2.

FIG. 8 shows a local sectional view of the support component of Example 2.

FIG. 9 shows a schematic diagram of theespiral electrode assembly of Example 3.

FIG. 10 shows a schematic diagram of a handle.

FIG. 11 shows a sectional view of a pressure sensor arrangement.

FIG. 12 shows a touch display screen of a device for radio frequency ablation.

FIG. 13 shows impedance detection values of a left lobe of a first swine lung at different handle grip strengths.

FIG. 14 shows impedance detection values of a right lobe of the first swine lung at different handle grip strengths.

FIG. 15 shows impedance detection values of a left lobe of a second swine lung at different handle grip strengths.

FIG. 16 shows impedance detection values of a right lobe of the second swine lung at different handle grip strengths.

FIG. 17 shows a relationship between the quantity of electrodes in contact and an impedance of an isolated swine lung.

FIG. 18 shows a relationship between the quantity of electrodes in contact with saline water and an impedance.

FIG. 19 shows detection values of radio frequency ablation on an impedance.

FIG. 20 shows a tissue impedance change curve of an animal test ablation process.

FIG. 21 shows tissue temperature and radio frequency output power curves of an ablation process without adopting segmentation control and temperature dynamic smoothing.

FIG. 22 shows tissue temperature and radio frequency output power curves of an ablation process after adoption of segmentation control and temperature dynamic smoothing.

FIG. 23 shows a record of stopped ablations during an ablation process in an animal test when a tissue temperature is higher than an over-temperature threshold value.

### DETAILED DESCRIPTION OF INVENTION

A device for radio frequency ablation of the present invention is able to generate and control a direct current, an alternating current and a radio frequency energy, collect, process and display a temperature, impedance or tension signal, and determine the effectiveness of an ablation according to the change of the impedance or tension signal, said change of the impedance is one or more parameters selected from the group consisting of fall in impedance, rate of change in impedance, a change in the rate of change in impedance, or a change from falling in impedance to rising in impedance. Further, the ablation is determined to be effective when said fall in impedance exceeds 10Ω to 100Ω, or the rate of change in impedance is higher than −1 Ω/s to −50 Ω/s, or the impedance changes from falling in impedance to rising in impedance.

The device for radio frequency ablation of the present invention uses a segmentation control method via a closed-loop control system to adjust a radio frequency output power so as to control an ablation temperature, said segmentation control comprises: (1) a fast heating phase: lasting for 0.5 s to 2 s from the beginning of ablation to reach a fast heating phase end point temperature that is 50% to 80% of the ablation temperature; (2) a slow heating phase: lasting for 0.5 s to 2 s after the fast heating phase to reach a slow heating phase end point temperature that is 70% to 99% of the ablation temperature, or is 0.1° C. to 10° C. lower than the ablation temperature; and (3) a stable maintenance phase: the temperature is stably maintained after the slow heating phase until the ablation is stopped.

At the same time, said device for radio frequency ablation performs dynamic smoothing on the temperature in the process of controlling the ablation temperature, comprising averaging, weighted averaging or median averaging sampled temperature values; said device for radio frequency ablation is guided to adjust the radio frequency power output based on said dynamic smoothed temperature values to ensure a smooth change of the radio frequency output power during the ablation process. An upper threshold value of the dynamic smoothing is 0.1° C./s to 20° C./s, and a lower threshold value is −0.1° C./s to −20° C./; when the temperature change rate is smaller than the lower threshold value, a smoothing time window is prolonged; when the temperature change rate is greater than the upper threshold value, the smoothing time window is shortened; and when the temperature change rate is between the lower and the upper threshold value, the smoothing time window remains unchanged. The upper threshold value of the dynamic smoothing is 5° C./s, and the lower threshold value is −5° C./s. The smoothing time window has a dynamic range of 0 s to 10 s, preferably 0 s to 2.5 s.

Further, said device for radio frequency ablation comprises a protection mechanism for preventing repeated ablation. The temperature of an ablation site is detected before each ablation. Ablation will not be performed if the temperature of the ablation site is higher than 40° C. to 60° C.

Further, said device for radio frequency ablation comprises a radio frequency energy delivery/feedback control mechanism: radio frequency energy is delivered to a tissue for 2 to 4 s so that said tissue reaches and maintains a set temperature for 6 to 8 s; an over-temperature alarm will be triggered and said device will stop delivery of radio frequency energy when temperature of said tissue is higher than an over-temperature threshold value. Said set temperature ranges from 60° C. to 70° C., and the over-temperature threshold value is 1° C. to 10° C. higher than the set temperature. Preferably, said set temperature is 65° C., and the over-temperature threshold value is 3° C. higher than the set temperature.

The objective of the present invention can be achieved by using the multi-electrode ablation device of an embodiment of the present invention. The following Examples are merely exemplary embodiments of the present invention and are not intended to limit the present invention in any way. Any simple amendments, equivalent variations and modifications made on the above embodiments according to the techniques and methods of the present invention are still within the scope of the techniques and methods of the solution of the present invention.

### EXAMPLE 1

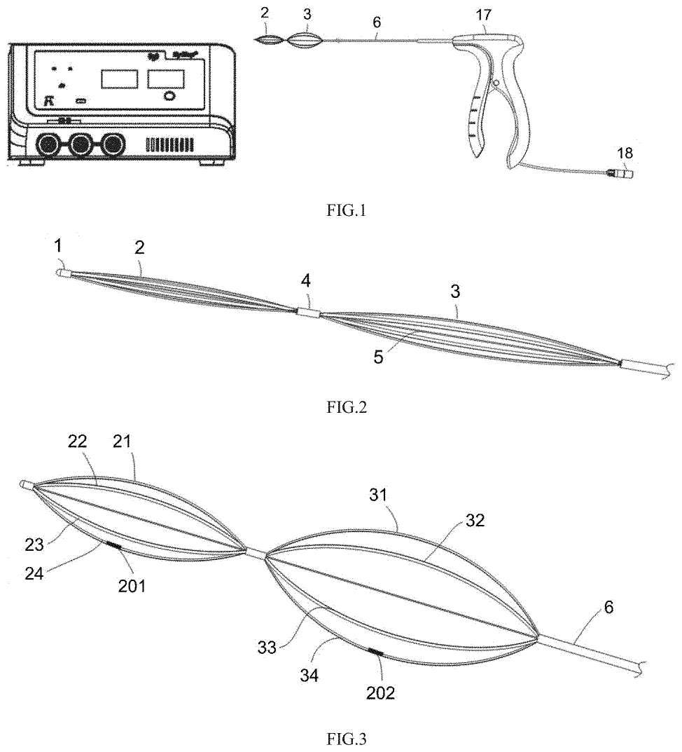

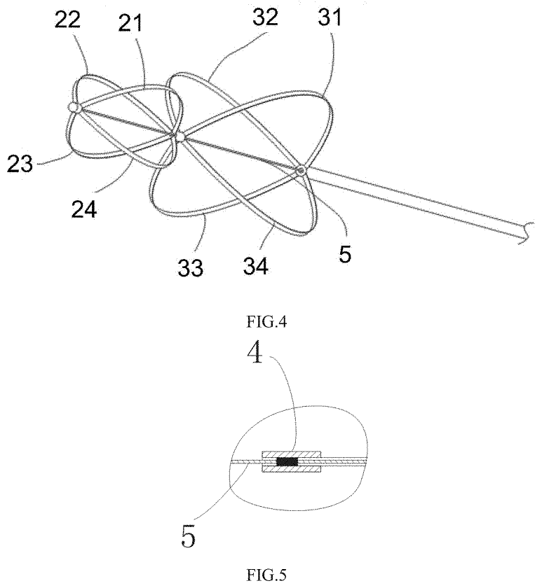

The present invention relates to a device for achieving a function of delivering energy in the trachea and bronchus, and further relates to a multi-electrode ablation device. As shown in FIG. 1, the device mainly comprises a first electrode assembly 2, a second electrode assembly 3, a guiding catheter body 6, a handle 17 and a connector 18. As shown in FIG. 2, the first electrode assembly 2 and the second electrode assembly 3 are continuously disposed in an axial direction of the guiding catheter body 6, and a damage-prevention structure 1 is disposed at a tip of the electrode assembly and is configured to fix the first electrode assembly 2 at the same time. The first electrode assembly 2 and the second electrode assembly 3 are connected to each other through a support component 4, a proximal end of the first electrode assembly 2 and a distal end of the second electrode assembly 3 are fixed to the support component 4, a distal end of a steel wire 5 is connected to the damage-prevention structure 1 at the tip, and a proximal end is fixed to the support component 4 (as shown in FIG. 5), and enters the handle 17 through the guiding catheter body 6. A proximal end of the second electrode assembly 3 is fixed to the catheter body 6. As shown in FIG. 3, when the handle 17 controls the steel wire 5 to contract towards the proximal end, the first electrode assembly 2 is driven to expand first, and at the same time, the second electrode assembly 3 synchronously expands according to the characteristics of the trachea tract, the electrode assembly is designed to be smaller at the distal end, and larger at the proximal end, with a diameter difference of about 1 to 5 mm.

The first electrode assembly 2 and the second electrode assembly 3 are provided with a plurality of electrodes: a first electrode 21, a second electrode 22, a third electrode 23, a fourth electrode 24. a fifth electrode 31, a sixth electrode 32, a seventh electrode 33 and an eighth electrode 34, the electrodes are made of stainless steel materials, and have certain elasticity, each electrode is electrically connected to the handle independently, and the handle is connected to a bronchial device for radio frequency ablation through the connector 18. When in use, each electrode forms a loop with a control circuit board through a trachea tissue, and each electrode can independently detect a contact impedance value between the electrode and the tissue. When the electrode is in good contact (the detected impedance value is 500Ω to 1000Ω), the bronchial device for radio frequency ablation will deliver radio frequency energy to ablate the lesion tissue, a temperature sensor 201 and a temperature sensor 202 are respectively disposed on the first electrode assembly 2 and the second electrode assembly 3, and can independently detect the temperature of the tissue around the corresponding electrode assembly.

### EXAMPLE 2

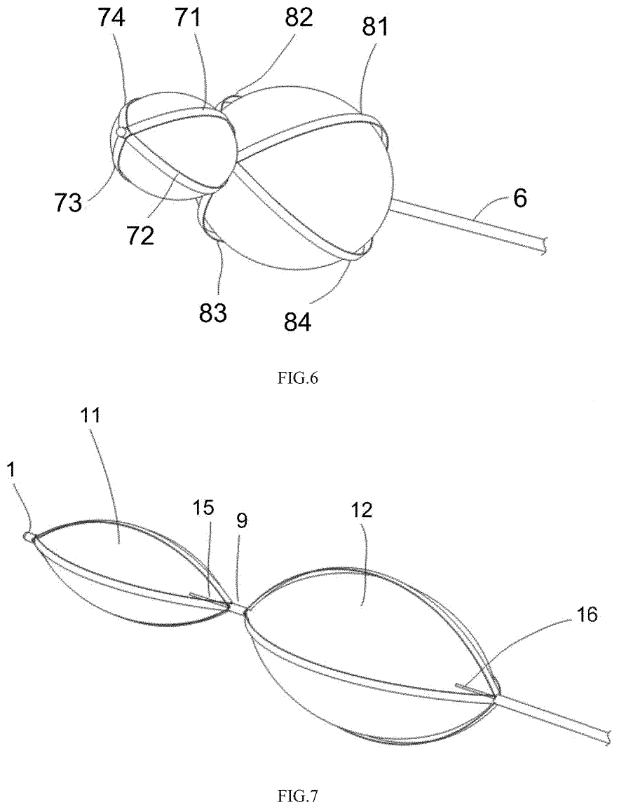

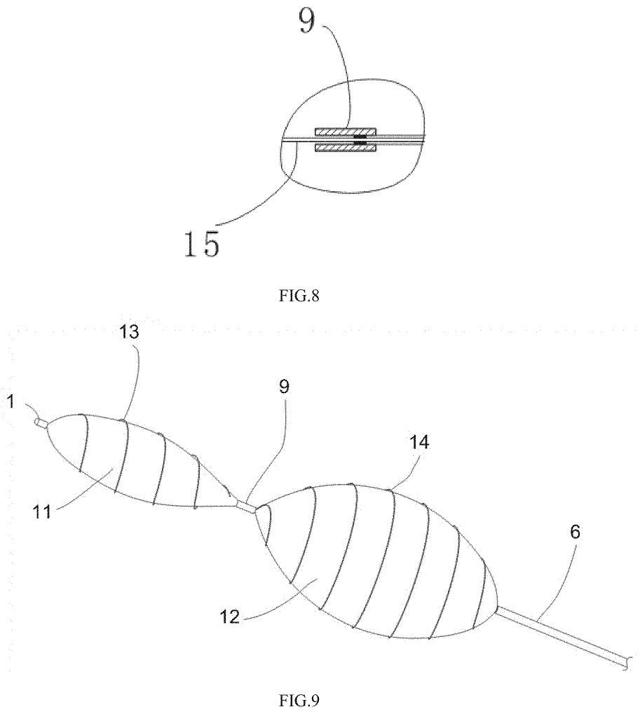

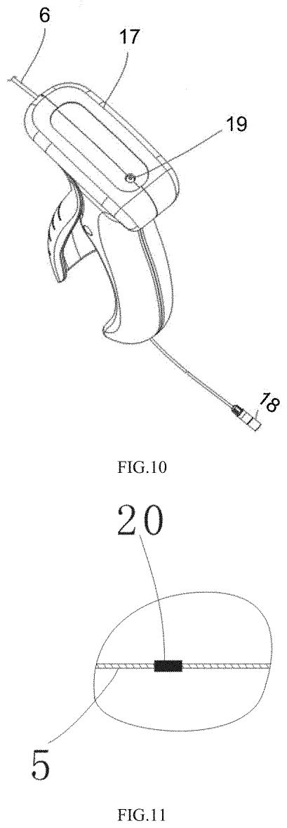

Devices as shown in FIG. 6 to FIG. 8 are a second embodiment of the present invention, a first balloon 11 and a second balloon 12 are disposed under the first electrode assembly 2 and the second electrode assembly 3, a proximal end of the first balloon 11 is provided with a first balloon air passage 15, and a proximal end of the second balloon 12 is provided with a second balloon air passage 16. The first balloon 11 and the second balloon 12 are isolated from each other, and the first air passage 15 and the second air passage 16 independently provide gas to the first balloon 11 and the second balloon 12. When the gas enters the balloons through the balloon air passages, a first electrode 71, a second electrode 72, a third electrode 73, a fourth electrode 74, a fifth electrode 81, a sixth electrode 82, a seventh electrode 83 and an eighth electrode 84 expand under pressure, the electrode assemblies expand, the gas inflow is controlled by an external gas inlet apparatus, the expansion size of the electrode assemblies can be set through the gas inflow, and the first electrode assembly 2 and the second electrode assembly 3 are independently controlled to adapt to the requirements of different sizes of the trachea lesion sites.

The first electrode 71, the second electrode 72, the third electrode 73. the fourth electrode 74, the fifth electrode 81, the sixth electrode 82, the seventh electrode 83 and the eighth electrode 84 are provided with independent conductive wires, When in use, each electrode forms a loop with a control circuit board through the trachea tissue, and each electrode can independently detect a contact impedance value between the electrode and the tissue. A temperature sensor 201 and a temperature sensor 202 are respectively disposed on the electrode assembly 2 and the electrode assembly 3, and can independently detect the temperature of the tissue around the corresponding electrode assembly.

### EXAMPLE 3

The device as shown in FIG. 9 is a third embodiment, a first annular electrode 1 and a second annular electrode 2 are spirally disposed on a first balloon 11 and a second balloon 12. When the balloons are inflated, outer diameters of the first annular electrode 1 and the second annular electrode 2 are increased. Independent conductive wires are disposed on the first annular electrode 1 and the second annular electrode 2. When in use, each electrode forms a loop with a control circuit board through a trachea tissue, and each electrode can independently detect a contact impedance value between the electrode and the tissue. A temperature sensor 201 and a temperature sensor 202 are respectively disposed on the annular electrode 1 and the annular electrode 2, and can independently detect the temperature of the tissue around the corresponding electrode assembly.

As shown in FIG. 10, an indicating lamp 19 is disposed on a handle 17. Theoretically, an impedance value of 500Ω to 1000Ω or below after the electrode is in contact with the tissue indicates that the radio frequency ablation can be performed. When a bronchial device for radio frequency ablation detects that the electrode contact impedance value is 500Ω to 1000Ω or below, the indicating lamp becomes green, indicating that the ablation can be performed When the bronchial device for radio frequency ablation detects that the electrode contact impedance value is 500Ω to 1000Ω or above, the indicating lamp is red, indicating that the discharging ablation cannot be performed.

As shown in FIG. 11, a pressure sensor 20 is disposed in a local area of a steel wire 5, two ends of the pressure sensor are respectively connected to two ends of the steel wire, when the electrode assembly is dragged, the steel wire 5 is stressed, and at this moment, the pressure sensor 20 will receive the same tension; through the treatment by the bronchial device for radio frequency ablation, the tension will be displayed to determine the level of contact. When the electrode is in contact with the tissue, the level of contact between an electrode arm and the tissue can be determined through determining the traction tension.

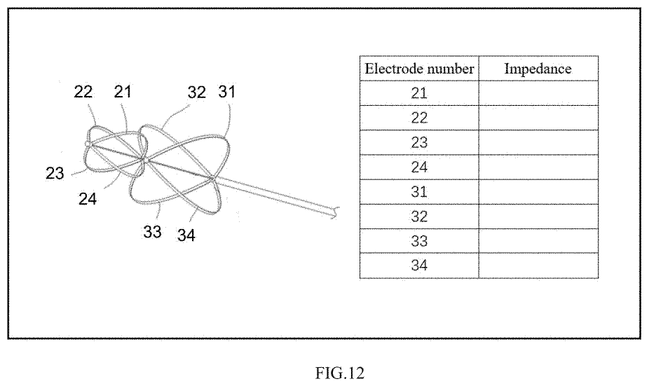

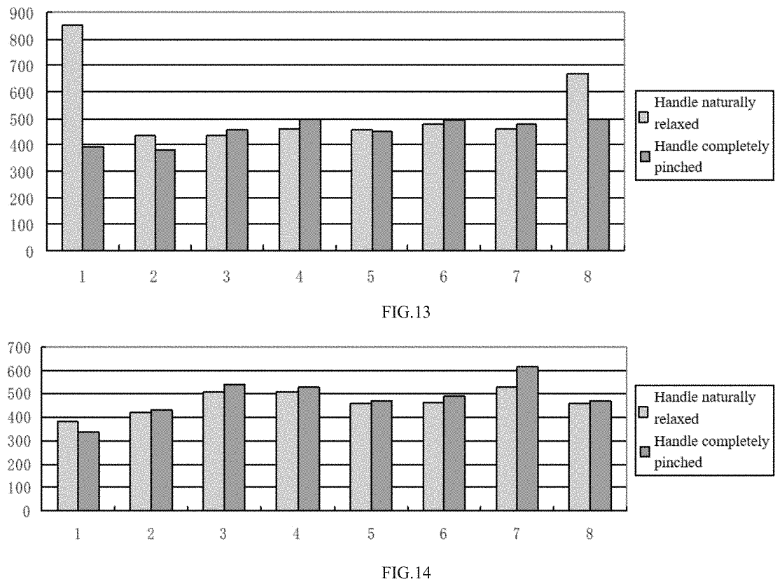

As shown in FIG. 12, the device for radio frequency ablation is provided with a touch display screen for displaying a status of the electrodes and a contact impedance value between the electrodes and a tissue, and energy can be delivered from one or more electrodes by clicking the touch display screen.

The guiding catheter body 6 can be served as a guiding tube, the guiding tube is provided with a tube lumen accommodating the electrode assembly 2 and the electrode assembly 3, the electrode assembles can freely extend and retract in the guiding tube, and liquids, such as anti-inflammatory medicine and anesthetics can enter the ablated lesion tissue through the tube lumen of the guiding tube so as to relive the pain and complications of a patient.

### EXAMPLE 4

### Investigation on Relationship Between the Impedance of Multi-Electrode Ablation Device of the Present Invention, and the Electrode Quantity and Tension

Clinical application of the multi-electrode ablation device was simulated through isolated tissue tests, and the impedance detection values of an ablation catheter under the conditions of different bronchus sites, different handle grip strengths and different quantities of electrodes in contact were observed.

Test environment: temperature: 15° C. to 20° C.; and humidity: 55% RH to 60% RH.

Test tissue: 2 fresh isolated swine lungs.

Test principle: the isolated swine lungs were soaked in saline water, the ablation catheter was connected onto the device for radio frequency ablation, the ablation catheter was operated, and the impedance display values on the device for radio frequency ablation were observed and recorded under the conditions of different bronchus sites, different handle grip strengths and different quantities of electrodes in contact.

Test sites: superior lobe of left lung, then inferior lobe of left lung, then superior lobe of right lung, and finally inferior lobe of right lung.

1. Investigation on Relationship Between Different Electrode Tension and Impedance

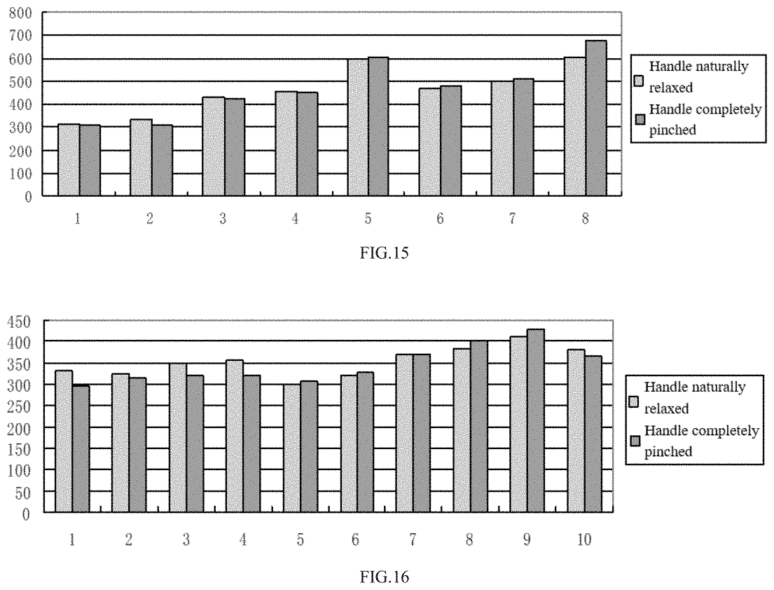

The impedance detection values under the conditions of the naturally relaxed state and the completely gripped state of the handle of the catheter at different bronchus sites are observed and recorded. The results are as shown in Tables 1 to 4 and FIGS. 13 to 16: The results show that the electrode tension is correlated with the impedance detection values.

TABLE 1
Impedance detection values of left lobe of the first swine lung
at different handle grip strengths

| | | Impedance (Ω) | | |
|---|---|---|---|---|
| Serial | | Handle naturally | Handle completely | Impedance |
| number | Test site | relaxed | gripped | change (Ω) |
| 1 | Superior lobe | 850 | 390 | 460 |
| | of left lung | | | |
| 2 | Superior lobe | 433 | 383 | 50 |
| | of left lung | | | |
| 3 | Superior lobe | 433 | 456 | −23 |
| | of left lung | | | |
| 4 | Superior lobe | 463 | 500 | −37 |
| | of left lung | | | |
| 5 | Inferior lobe | 453 | 448 | 5 |
| | of left lung | | | |
| 6 | Inferior lobe | 478 | 494 | −16 |
| | of left lung | | | |
| 7 | Inferior lobe | 461 | 478 | −17 |
| | of left lung | | | |
| 8 | Inferior lobe | 671 | 496 | 175 |
| | of left lung | | | |

TABLE 2
Impedance detection values of right lobe of the first swine lung
at different handle grip strengths

| | | Impedance (Ω) | | |
|---|---|---|---|---|
| | | Handle | Handle | Impedance |
| Serial | | naturally | completely | change |
| number | Test site | relaxed | gripped | (Ω) |
| 1 | Superior lobe of | 380 | 340 | 40 |
| | right lung | | | |
| 2 | Superior lobe of | 418 | 430 | −12 |
| | right lung | | | |
| 3 | Superior lobe of | 507 | 540 | −33 |
| | right lung | | | |
| 4 | Superior lobe of | 512 | 530 | −18 |
| | right lung | | | |
| 5 | Inferior lobe of | 460 | 470 | −10 |
| | right lung | | | |
| 6 | Inferior lobe of | 467 | 490 | −23 |
| | right lung | | | |
| 7 | Inferior lobe of | 530 | 620 | −90 |
| | right lung | | | |
| 8 | Inferior lobe of | 460 | 470 | −10 |
| | right lung | | | |

TABLE 3
Impedance detection values of left lobe of the second swine
lung at different handle grip strengths

| | | Impedance (Ω) | | |
|---|---|---|---|---|
| | | Handle | Handle | Impedance |
| Serial | | naturally | completely | change |
| number | Test site | relaxed | gripped | (Ω) |
| 1 | Superior lobe of | 315 | 305 | 10 |
| | left lung | | | |
| 2 | Superior lobe of | 334 | 305 | 29 |
| | left lung | | | |
| 3 | Superior lobe of | 428 | 425 | 3 |
| | left lung | | | |
| 4 | Superior lobe of | 458 | 450 | 8 |
| | left lung | | | |
| 5 | Interior lobe of | 596 | 601 | −5 |
| | left lung | | | |
| 6 | Inferior lobe of | 467 | 480 | −13 |
| | left lung | | | |
| 7 | Inferior lobe of | 496 | 510 | −14 |
| | left lung | | | |
| 8 | Inferior lobe of | 604 | 678 | −74 |
| | left lung | | | |

TABLE 4
Impedance detection values of right lobe of the second swine
lung at different handle grip strengths

| | | Impedance (Ω) | | |
|---|---|---|---|---|
| | | Handle | Handle | Impedance |
| Serial | | naturally | completely | change |
| number | Test site | relaxed | gripped | (Ω) |
| 1 | Superior lobe of | 330 | 298 | 32 |
| | right lung | | | |
| 2 | Superior lobe of | 326 | 315 | 11 |
| | right lung | | | |
| 3 | Superior lobe of | 350 | 320 | 30 |
| | right lung | | | |
| 4 | Superior lobe of | 355 | 320 | 35 |
| | right lung | | | |
| 5 | Inferior lobe of | 300 | 308 | −8 |
| | right lung | | | |
| 6 | Inferior lobe of | 320 | 329 | −9 |
| | right lung | | | |
| 7 | Inferior lobe of | 370 | 370 | 0 |
| | right lung | | | |
| 8 | Inferior lobe of | 384 | 400 | −16 |
| | right lung | | | |
| 9 | Inferior lobe of | 410 | 428 | −18 |
| | right lung | | | |
| 10 | Inferior lobe of | 380 | 365 | 15 |
| | right lung | | | |

2. Investigation on Relationship Between Different Quantities of Electrodes in Contact and Impedance

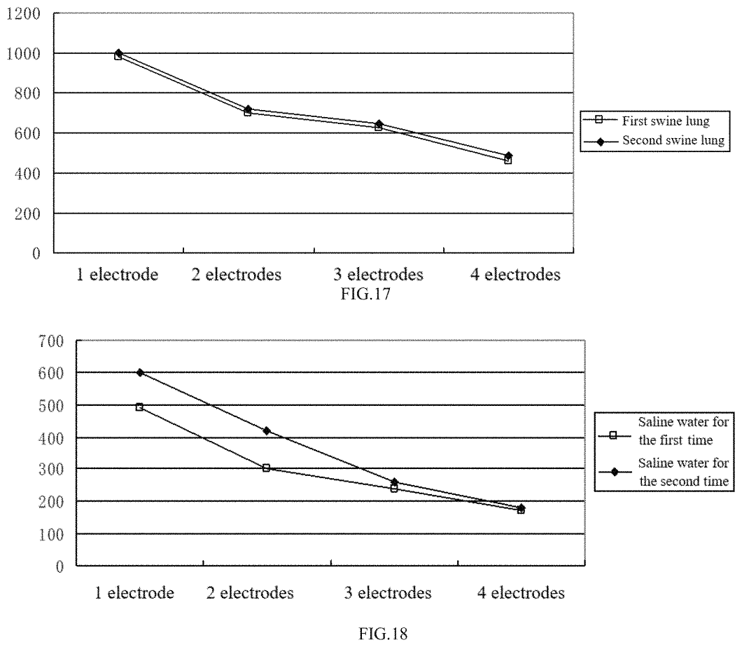

Different quantities of electrodes are in contact with the bronchus, the impedance detection values are observed and recorded, and the results are as shown in Table 5 and FIG. 17. Different quantities of electrodes are soaked into saline water, the impedance detection values are observed and recorded (with the influence of the contact pressure excluded), and the results are as shown in Table 6 and FIG. 18, The results show that different quantities of electrodes in contact have obvious influence on the impedance detection values, the greater the number of electrodes in contact, the smaller the impedance detection value, and the quantity of electrodes in contact can be determined according to the impedance detection value.

TABLE 5
Relationship between quantity of electrodes in contact and
impedance of isolated swine lung

| | Impedance (Ω) | | | |
|---|---|---|---|---|
| Serial number | 1 electrode | 2 electrodes | 3 electrodes | 4 electrodes |
| The first swine | 980 | 700 | 630 | 460 |
| lung | | | | |
| The second | 999 | 720 | 650 | 490 |
| swine lung | | | | |

TABLE 6
Relationship between quantity of electrodes in contact and
impedance of saline water

| | Impedance (Ω) | | | |
|---|---|---|---|---|
| Serial number | 1 electrode | 2 electrodes | 3 electrodes | 4 electrodes |
| Saline water for | 490 | 300 | 240 | 170 |
| the first time | | | | |
| Saline water for | 600 | 420 | 260 | 180 |
| the second time | | | | |

3. Investigation on Influence of Radio Frequency Ablation on Impedance

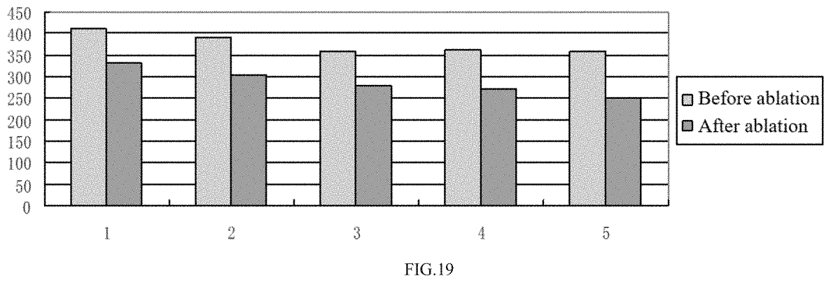

The radio frequency is output, the impedance detection values are observed and recorded, the results are as shown in Table 7 and FIG. 19, and the results show that the radio frequency ablation causes impedance detection value to fall; the effectiveness of the ablation can be determined according to the change of an impedance or tension signal, and the change of the impedance is one or more parameters selected from a group consisting of fall in value of impedance, rate of change in impedance, the change in the rate of change in impedance, or the change from falling in impedance to rising in impedance.

TABLE 7
Detection values of radio frequency ablation on impedance

| | | Impedance (Ω) | | | Highest |
|---|---|---|---|---|---|
| Serial | Test | Before | After | Impedance | temperature |
| number | conditions | ablation | ablation | change (Ω) | (° C.) |
| 1 | 18 W, 10 s | 411 | 330 | 81 | 50 |
| 2 | 18 W, 10 s | 392 | 302 | 90 | 60 |
| 3 | 18 W, 10 s | 360 | 279 | 81 | 67 |
| 4 | 18 W, 15 s | 363 | 272 | 91 | 80 |
| 5 | 65° C., 15 s | 360 | 252 | 108 | 66 |

### EXAMPLE 5

### Investigation on Effectiveness of Ablation of the Multi-Electrode Ablation Device of Present Invention

The effectiveness of an ablation of the multi-electrode ablation device of the present invention is investigated by using an animal test. Through defining a logic relationship among the impedance, power and temperature, the generated and controlled direct current, alternating current and radio frequency energy are precisely controlled; a temperature or impedance signal is collected, processed and displayed; and the effectiveness of the ablation is determined according to the change of the impedance signal. The ablation was determined to be effective when a fall in impedance exceeded 10Ω to 100Ω, or a rate of change in impedance is higher than −1 Ω/s to −50 Ω/s, or the impedance changes from falling in impedance to rising in impedance.

Specific operations are as follows:

Electrodes of the multi-electrode ablation device of the present invention was put into a site of a dog lung to be tested, and a data interface of the multi-electrode ablation device was connected to a computer. The multi-electrode ablation device was operated for ablation. The computer displayed and recorded the temperature, power and impedance data in the test process. A bronchial endoscope was used to observe the entire test process.

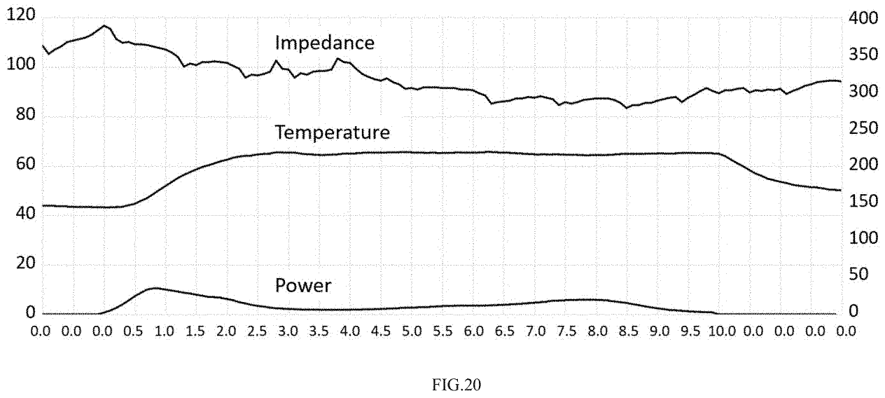

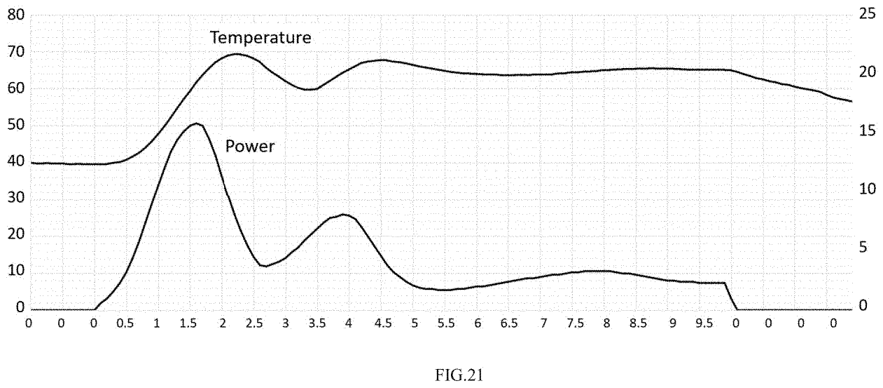

The results are as shown in FIG. 20. FIG. 20 is a tissue impedance change curve of an ablation process in animal tests. The abscissa is the time, the left ordinate is the tissue temperature and the radio frequency output power, and the right ordinate is the tissue impedance. As shown in the figure, after the ablation is started, the tissue impedance starts to fall; additionally, the tissue impedance falling speed was gradually decelerated, and then the tissue impedance gradually starts to rise, indicating that the ablation of the multi-electrode ablation device of the present invention is effective.

### EXAMPLE 6

### Investigation on Safety and Temperature Anti-Interference Capability of the Multi-Electrode Ablation Device of Present Invention

The present invention relates to a device with a function of delivering energy in the trachea and bronchus, and the device uses a segmentation proportional integral control algorithm to perform dynamic smoothing on the temperature. 0 s to 1 s from the beginning of the ablation is a fast heating phase, the radio frequency output power rises quickly from 0 to above 10 W, and the tissue temperature starts to rise quickly. 1 s to 2 s is a slow heating phase, the radio frequency Output power slowly rises, and starts to gradually fall, and the tissue temperature heating speed starts decelerate. After 2 s till the ablation stops is a stable maintenance phase, and the radio frequency output power slowly falls and is adjusted slightly so as to maintain the tissue temperature.

The temperature dynamic smoothing time window has a dynamic range of 0 s to 2.5 s. Each time when a temperature change rate is greater than 5° C./s, the smoothing time window is shortened by 0.01 s. Each time when the temperature change rate is smaller than −5° C./s, the smoothing time window is prolonged by 0.01 s, When the temperature change rate is between −5° C./s and 5° C./s, the smoothing time window remains unchanged. The temperature in the smoothing time window is subjected to an average calculation, thus achieving the temperature dynamic smoothing.

The operations of the animal test are the same as those in Example 5.

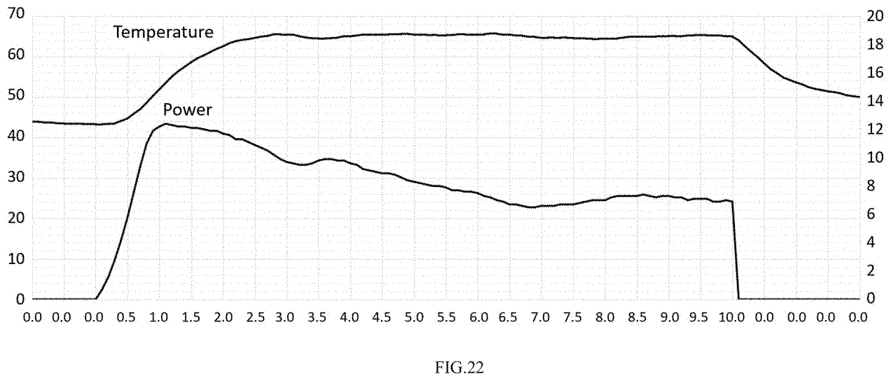

The results are shown in FIG. 21 and FIG. 22. FIG. 21 shows tissue temperature and radio frequency output power curves of an ablation process without adopting segmentation control and temperature dynamic smoothing in the animal test. FIG. 22 shows tissue temperature and radio frequency output power curves of an ablation process after adoption of segmentation control and temperature dynamic smoothing. The abscissa is the time, the left ordinate is the tissue temperature, and the right ordinate is the radio frequency output power. As shown in the figure, after the ablation is started, the radio frequency output power rises quickly within 1 s, slowly rises and starts to fall within 2 s, and slowly falls and is adjusted slightly after 2 s. After the ablation is started, the tissue temperature starts to rise quickly within 1 s, slowly rises within 2 s, and reaches the ablation temperature within 3 s and maintains at the ablation temperature. The device controlled the radio frequency output power so that the temperature of the ablation electrodes reached the ablation temperature within 3 s. Additionally, after the ablation temperature is reached, the temperature overshoot is less than 1° C., the tissue temperature is stably maintained at the ablation temperature, and the fluctuation is smaller than 1° C., In the whole ablation treatment process, the radio frequency output power smoothly changes without suddenly applied and (or) suddenly changed radio frequency energy. When the segmentation control and temperature dynamic smoothing are not adopted, the tissue temperature generates obvious oscillation, and the temperature overshoot is greater. After the segmentation control and temperature dynamic smoothing are adopted, the tissue temperature is kept stable, and the temperature overshoot is smaller.

The results shows that the radio frequency output power is successfully adjusted by using the closed loop control system through the segmentation control method to control the ablation temperature, and the temperature dynamic smoothing is utilized to overcome various kinds of disturbances. Therefore, the safety and the effectiveness of the system are further ensured, i.e., the situations of incorrect ablation or ablation incapability cannot occur, and the situations of repeated ablation or excessive ablation also cannot occur.

### EXAMPLE 7

### Investigation on Safety Control Capability of the Radio Frequency Ablation Device of Present Invention

The radio frequency ablation device of the present invention comprises a radio frequency energy delivery/feedback control mechanism: radio frequency energy is delivered to a tissue for 2 to 4 s so that said tissue reached a set temperature of 60° C. to 70° C. and is maintained for 6 to 8 s; an over-temperature alarm will be triggered and said device will stop delivery of radio frequency energy when temperature of said tissue is higher than an over-temperature threshold value (1° C. to 10° C. higher than the set temperature).

The operations of the animal test are the same as those in Example 5.

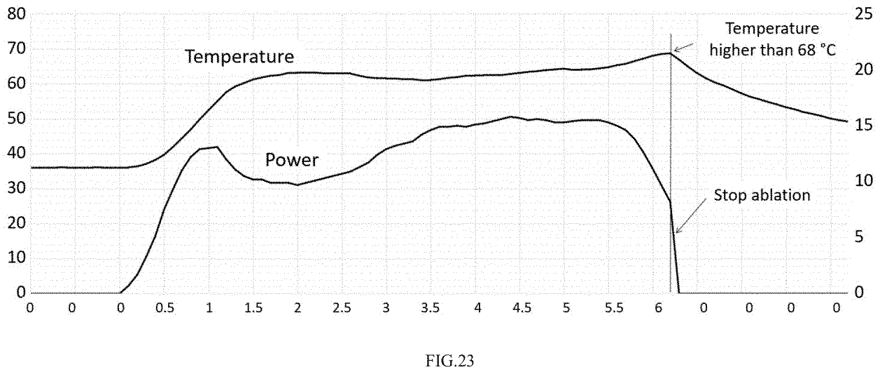

The results are as shown in FIG, 23. FIG. 23 shows a record of ablation stopping during an ablation process in an animal test, when the tissue temperature is higher than the over-temperature threshold value. The abscissa is the time, the left ordinate is the tissue temperature, and the right ordinate is the radio frequency output power. As shown in the figure, when the tissue temperature is higher than 68° C., the radio frequency output power falls rapidly to 0, and the ablation is stopped

1-45. (canceled)

46. A device for radio frequency ablation, for delivering energy in the trachea and bronchus, characterized in that: said device being able to generate and control direct current, alternating current and radio frequency energy; collect, process and display temperature, impedance or tension signal; and determine effectiveness of an ablation. according to change of impedance, said change of impedance is one or more parameters selected from the group consisting of fall in impedance, rate of change in impedance, a change in rate of change in impedance, and a change from falling in impedance Co rising in impedance,
wherein said ablation is determined Co be effective when said fall in impedance exceeds 10Ω to 100Ω, or said rate of change in impedance is higher than −1 Ω/s to −50 Ω/s, or said impedance changes from failing in impedance Co rising in impedance,
wherein said device for radio frequency ablation uses a segmentation control method via a closed-loop control system to adjust a radio frequency output power so as to control an ablation temperature, said segmentation control comprises: (1) a fast heating phase: lasting for 0.5 s to 2 s from the beginning of ablation to reach a fast heating phase end point temperature that is 50% to 80% of said ablation temperature; (2) a slow heating phase: lasting for 0.5 s to 2 s after said fast heating phase to reach a slow heating phase end point temperature that is 70% to 99% of said ablation. temperature, or is 0.1° C. to 10° C. lower than said ablation temperature; and (3) a stable maintenance phase: temperature is stably maintained after said slow heating phase until the ablation is stopped.

47. The device for radio frequency ablation of claim 46, wherein the ablation is determined to be effective when said fall in impedance exceeds 2Ω to 50Ω, or said rate of change in impedance is higher than −5 Ω/s to −50 Ω/s, or said impedance changes from falling in impedance to rising in impedance.

48. The device for radio frequency ablation of claim 46, wherein said segmentation control comprises: (1) said fast heating phase: lasting for 1 s from the beginning of ablation, wherein said fast heating phase end point temperature is 65% of said ablation temperature; (2) said slow heating phase: lasting for 1 s after said fast heating stage, wherein said slow heating phase end point temperature is 90% of said ablation temperature, or is 2° C. lower than said ablation temperature; and (3) the stable maintenance phase: temperature is stably maintained after said slow heating phase until the ablation is stopped.

49. The device for radio frequency ablation of claim 46, wherein said device for radio frequency ablation performs dynamic smoothing on the temperature during control process of said ablation temperature to obtain dynamic smoothed temperature values, comprising averaging, weighted averaging or median averaging sampled temperature values; said device for radio frequency ablation is guided to adjust the radio frequency power output based on said dynamic smoothed temperature values to ensure smooth change of the radio frequency output power during the ablation process.

50. The device for radio frequency ablation of claim 49, wherein an upper threshold value of the dynamic smoothing is 0.1° C./s to 20° C./s, and a lower threshold value is −0.1° C./s to −20° C./; when a temperature change rate is smaller than the lower threshold value, a smoothing time window is prolonged; when the temperature change rate is greater than the upper threshold value, the smoothing time window is shortened; and when the temperature change rate is between the lower and the upper threshold value, the smoothing time window remains unchanged.

51. The device for radio frequency ablation of claim 50, wherein said upper threshold value of said dynamic smoothing is 5° C./s, and said lower threshold value is −5° C./s.

52. The device for radio frequency ablation of claim 50, wherein the smoothing time window has a dynamic range from 0 s to 10 s.

53. The device for radio frequency ablation of claim 52, wherein the smoothing time window has a dynamic range from 0 s to 2.5 s.

54. The device for radio frequency ablation of claim 46, further comprising a protection mechanism for preventing repeated ablation, wherein temperature of an ablation site is detected before each ablation, and ablation will not be performed if the temperature of said ablation site is higher than 40° C. to 60° C.

55. The device for radio frequency ablation of claim 54, wherein temperature of an ablation site is detected before each ablation, and ablation will not be performed if temperature of said ablation site is higher than 45° C.

56. The device for radio frequency ablation of claim 46, further comprising using one or both of the following methods: Method 1) detecting the impedance by a continuous weak alternating current signal, and calculating the impedance through a voltage and a current during radio frequency output; Method 2) directly detecting the impedance without radio frequency output.

57. The device for radio frequency ablation of claim 46, comprising a radio frequency energy delivery/feedback control mechanism, wherein: radio frequency energy is delivered to a tissue for 2 to 4 s so that said tissue reaches and maintains a set temperature for 6 to 8 s; an over-temperature alarm will be triggered and said device will stop delivery of radio frequency energy when temperature of said tissue is higher than an over-temperature threshold value.

56. The device for radio frequency ablation of claim 57, wherein said set temperature ranges from 60° C. to 70° C., and said over-temperature threshold value is 1° C. to 10° C. higher than said set temperature.

59. The device for radio frequency ablation of claim 56, wherein said set temperature is 65° C., and said over-temperature threshold value is 3° C. higher than said set temperature.

60. The device for radio frequency ablation of claim 46, wherein said device for radio frequency ablation adopts a design of multiple central controllers, dual circuit design for temperature, voltage and current.

61. The device for radio frequency ablation of claim 46, wherein the device for radio frequency ablation is provided with a data transmission interface for external connection to a computer to obtain information of various parameters in real time.

62. The device for radio frequency ablation of claim 46, wherein said device for radio frequency ablation is provided with a touch display screen for displaying a status of the electrodes and a contact impedance value between the electrodes and a tissue, and energy can be delivered from one or more electrodes by clicking the touch display screen.

63. A multi-electrode ablation device, comprising the device for radio frequency ablation of claim 46, electrode assemblies, a guiding catheter, a handle and a connector, wherein said guiding catheter comprises at least one lumen;
said electrode assemblies are disposed at a front end of said guiding catheter, and are connected to said handle through circuitries inside said guiding catheter, said electrode assemblies comprise more than one electrode groups and more than one detection devices, said electrode groups are able to deliver electric energy, radio frequency energy, laser energy, high-density focused ultrasound or low temperature for ablation, and said detection devices are configured to detect temperature, impedance or tension;
said handle is connected to said connector and said electrode assemblies, and comprises one or more operation components, said operation components are configured to control constriction, expansion and energy release of the electrode groups and are able to control the electrode assemblies to extend out of or retreat back into the guiding catheter; and
said connector is configured to provide an energy to the electrodes.

64. The multi-electrode ablation device of claim 63, wherein said detection devices comprise a temperature detection device, an impedance detection device and a tension detection device.

65. The multi-electrode ablation device of claim 64, wherein said electrode groups comprise one or more electrodes, each electrode is electrically connected to said handle independently, said electrode groups expands in a basket shape, spiral shape or balloon shape under the control of said operation components, and under the presence of more than one electrode group, said electrode groups are sequentially arranged in series with electrode groups closer to said handle having a larger outer diameter after expansion, wherein said outer diameter is from 1 to 20 mm.

66. The multi-electrode ablation device claim wherein said electrode assemblies further comprise a steel wire, each of said electrodes comprises two ends, each of said two ends is fixed to said steel wire, said steel wire passes through the guiding catheter to be connected to said handle, and said handle controls the contraction and expansion of said electrode groups by pulling and releasing said steel wire.

67. The multi-electrode ablation device of claim 66, wherein under the presence of more than one electrode group, a damage-prevention structure is disposed at a tip of an electrode group that is most distal from the handle among said electrode groups, and the electrode groups are connected to one another through support components.

68. The multi-electrode ablation device of claim 66, wherein a pressure sensor is disposed on said steel wire.

69. The multi-electrode ablation device of claim 65, wherein said electrode assemblies further comprise a balloon, said balloon is disposed between the electrodes, said balloon is connected to said handle via a balloon air passage which passes through said guiding catheter, said balloon is adapted for connection to a gas inlet apparatus through said handle, and said electrode groups expand after the balloon is inflated; under the presence of more than one electrode group, more than one balloons are sequentially arranged in series, and are respectively connected to said handle through independent balloon air passages.

70. The multi-electrode ablation device claim. 63, characterized in that: hardness of said guiding catheter increases with proximity to said handle, said hardness ranges from 90 A to 80 D on the Shore hardness scale.

71. The multi-electrode ablation device of claim 63, wherein the operation component of said handle comprises a control circuit board and a control button, said control circuit board is connected to the electrode assemblies and the control button, and said control button controls different components in different electrode assemblies respectively.

72. The multi-electrode ablation device of claim 65, wherein said operation component of said handle controls said electrode groups so as to control said one or more electrodes for energy delivery.

73. The multi-electrode ablation device of claim 63, wherein said device for radio frequency ablation displays an impedance or tension of the electrodes and indicate whether said electrode assemblies are in good contact with the tracheal wall: an impedance value smaller than or equal to a threshold value of impedance after the electrodes are in contact with a tissue indicates good contact between said electrode assemblies and said tracheal wall.

74. The multi-electrode ablation device of claim 73, wherein said threshold value of impedance ranges from 500Ω to 1000Ω.

75. The multi-electrode ablation device of claim 74, wherein said threshold value of impedance is 900Ω.

76. The multi-electrode ablation device of claim 63, wherein said device for radio frequency ablation determines whether said electrodes are is good contact with the tracheal wall, comprising the steps of: measuring the impedance of each electrode using said device for radio frequency ablation; the impedances are consistent, the contact between the electrodes and the tracheal wall is good; if the contact between a certain electrode and the tracheal wall is not good, the impedance will be different from that of others in good contact.
